(19) United States
(12) Patent Application Publication
(10) Pub. No.: US 2005/0019308 A1
(43) Pub. Date: Jan. 27, 2005

(54) Oncolytic viruses for the treatment of neoplasms having activated PP2A or Rac

(75) Inventors: Kara L. Norman, Menlo Park, CA (US); Patrick W. K. Lee, Halifax (CA)

Correspondence Address: FISH & RICHARDSON P.C., 3300 DAIN RAUSCHER PLAZA, MINNEAPOLIS, MN, 55402, US

(73) Assignee: Oncolytics Biotech Inc., Calgary (CA)

(21) Appl. No.: 10/886,022

(22) Filed: Jul. 6, 2004

Related U.S. Application Data
(60) Provisional application No. 60/484,643, filed on Jul. 7, 2003.

(51) Int. Cl. A61K 48/00

(52) U.S. Cl. 424/93.2; 424/93.6

(57) ABSTRACT

Methods for treating neoplasms, by administering oncolytic viruses to a neoplasm having activated PP2A-like or Rac activities, are disclosed. The virus is administered so that it ultimately directly contacts target cancer cells. Combinations of more than one type and/or strain of oncolytic viruses can be used. Of particular interest is the use of reovirus.

### RELATED APPLICATION

This application claims the benefit of U.S. Provisional Application No. 60/484,643, filed Jul. 7, 2003, which is hereby incorporated by reference in its entirety.

### TECHNICAL FIELD

The present invention pertains to methods for treating neoplasms in an animal using oncolytic viruses. A particular embodiment is reovirus. In particular, the neoplasms have elevated protein phosphatase 2A-like activities or an activated Rac pathway.

### REFERENCES

U.S. Pat. No. 5,023,252.
U.S. Pat. No. 6,136,307.
Bialojan, C., and Takai, A. (1988). Inhibitory effect of a marine-sponge toxin, okadaic acid, on protein phosphatases. Specificity and kinetics. Biochem J. 256(1):283-290.
Chandran, K., and Nibert, M. L. (1998). Protease cleavage of reovirus capsid protein mul/mulC is blocked by alkyl sulfate detergents, yielding a new type of infectious subvirion particle. J. Virol. 72:467-475.
Chang, H. W., et al. (1992). The E3L gene of vaccinia virus encodes an inhibitor of the interferon-induced, double-stranded RNA-dependent protein kinase. Proc Natl Acad Sci USA 89(11):4825-4829.
Chang, H. W., and Jacobs, B. L. (1993). Identification of a conserved motif that is necessary for binding of the vaccinia virus E3L gene products to double-stranded RNA. Virology 194(2):537-547.
Chang, H. W., et al. (1995). Rescue of vaccinia virus lacking the E3L gene by mutants of E3L. J. Virol. 1995 69(10):6605-6608.
Cuff, C. F., et al. (1998). Enteric reovirus infection as a probe to study immunotoxicity of the gastrointestinal tract. Toxicological Sciences 42:99-108.
Duncan, R., et al. (1991). Conformational and functional analysis of the C-terminal globular head of the reovirus cell attachment protein. Virology 182:810-819.
Fields, B. N., et al. (1996). Fundamental Virology, 3rd Edition, Lippincott-Raven.
Goodman and Gilman's The Pharmacological Basis of Therapeutics by Gilman, A. G., Goodman, L. S., Rall, T. W., and Murad, F. (1985). Seventh Edition, MacMillan Press, N.Y.
Kawagishi-Kobayashi, M., et al. (1997). Regulation of the protein kinase PKR by the vaccinia virus pseudosubstrate inhibitor K3L is dependent on residues conserved between the K3L protein and the PKR substrate eIF2alpha. Mol Cell Biol. 17(7):4146-4158.
Lee, J. M., and Bernstein, A. (1993). p53 mutations increase resistance to ionizing radiation. Proc Natl Acad Sci USA. 90(12):5742-5746.
Lowe, S. W., et al. (1994). p53 status and the efficacy of cancer therapy in vivo. Science. 266(5186):807-810.
Mah, D. C., et al. (1990). The N-terminal quarter of reovirus cell attachment protein sigma 1 possesses intrinsic virion-anchoring function. Virology 179:95-103.
Mills, J. C., et al. (1998). Activation of a PP2A-like phosphatase and dephosphorylation of tau protein characterize onset of the execution phase of apoptosis. J Cell Sci. 111 (Pt 5):625-636.
Nedergaard, T., et al. (1997). A one-step DGGE scanning method for detection of mutations in the K-, N-, and H-ras oncogenes: mutations at codons 12, 13 and 61 are rare in B-cell non-Hodgkin's lymphoma. Int. J. Cancer 71:364-369.
Nibert, M. L., et al. (1995). Reoviruses and their replication in Fields Virology, 3rd Edition, Lippincott-Raven Press, pp. 1557-1596.
Raybaud-Diogene, H., et al. (1997). JMarkers of radioresistance in squamous cell carcinomas of the head and neck: a clinicopathologic and immunohistochemical study. J Clin Oncol. 15(3):1030-1038.
Romano, P. R., et al. (1998). Inhibition of double-stranded RNA-dependent protein kinase PKR by vaccinia virus E3: role of complex formation and the E3 N-terminal domain. Mol Cell Biol. 18(12):7304-7316.
Scita, G., et al. (2000). Signaling from Ras to Rac and beyond: not just a matter of GEFs. EMBO J. 19(11):2393-2398.
Sharp, T. V., et al. (1998). The vaccinia virus E3L gene product interacts with both the regulatory and the substrate binding regions of PKR: implications for PKR autoregulation. Virology. 250(2):302-315.
Tian, L., et al. (1998). Glucocorticoid regulation of calcium-activated potassium channels mediated by serine/threonine protein phosphatase. J. Biol. Chem. 273(22):13531-13536.
Toumebize, R., et al. (1997). Distinct roles of PP1 and PP2A-like phosphatases in control of microtubule dynamics during mitosis. EMBO J. 16(18):5537-5549.
Turner, D. L., et al. (1992). Site directed mutagenesis of the C-terminal portion of reovirus protein sigma 1: evidence for a conformation-dependent receptor binding domain. Virology 186:219-227.

All of the publications, patent applications and patents cited above or elsewhere in this application are herein incorporated by reference in the entirety to the same extent as if each individual publication, patent application or patent is specifically and individually indicated to be incorporated by reference in its entirety.

### BACKGROUND

Normal cell proliferation is regulated by a balance between growth-promoting proto-oncogenes and growth-constraining tumor-suppressor genes. Tumorigenesis can be caused by genetic alterations to the genome that result in the mutation of those cellular elements that govern the interpretation of cellular signals, such as potentiation of proto-oncogene activity or inactivation of tumor suppression. It is believed that the interpretation of these signals ultimately influences the growth and differentiation of a cell, and that misinterpretation of these signals can result in neoplastic growth (neoplasia).

Neoplasms are mainly treated by conventional therapies including surgery, chemotherapy, and radiation. Surgery is typically used as the primary treatment for early stages of cancer; however, many tumors cannot be completely removed by surgical means. In addition, metastatic growth of neoplasms may prevent the complete cure of cancer by surgery. Chemotherapy involves administration of compounds having antitumor activity, such as alkylating agents, antimetabolites, and antitumor antibiotics. The efficacy of chemotherapy is often limited by severe side effects, including nausea and vomiting, bone marrow depression, renal damage, and central nervous system depression. Radiation therapy relies on the greater ability of normal cells, in contrast with neoplastic cells, to repair themselves after treatment with radiation. Radiotherapy cannot be used to treat many neoplasms, however, because of the sensitivity of tissue surrounding the tumor. In addition, certain tumors have demonstrated resistance to radiotherapy, and such may be dependent on the oncogene or anti-oncogene status of the cell (Lee et al., 1993; Lowe et al., 1994; Raybaud-Diogene et al., 1997). In view of the drawbacks associated with the current means for treating neoplastic growth, the need still exists for improved methods for the treatment of neoplasms.

### SUMMARY

The present invention provides a method of treating a neoplasm in an animal by using an oncolytic virus, particularly a reovirus, wherein the neoplasm has an activated Rac pathway or elevated PP2A-like phosphatase activities. Also provided are methods of determining if a neoplasm is susceptible to reovirus (or similar oncolytic viruses) infection by measuring the activities of PP2A-like phosphatase or the Rac pathway.

Accordingly, one aspect of the present invention provides a method of treating or ameliorating a neoplasm in an animal, comprising administering to the animal an effective amount of one or more reoviruses under conditions which result in substantial lysis of neoplastic cells in the neoplasm, wherein the neoplasm comprises elevated PP2A-like phosphatase activities or an activated Rac pathway.

The animal may be a mammal, particularly one selected from the group consisting of dogs, cats, sheep, goats, cattle, horses, pigs, humans, and non-human primates. The animal is preferably human. The animal may be immunocompetent.

The neoplasm may be a solid neoplasm, including but not limited to, lung cancer, prostate cancer, colorectal cancer, thyroid cancer, renal cancer, adrenal cancer, liver cancer, pancreatic cancer, breast cancer, or central or peripheral nervous system cancer. In another embodiment, the neoplasm is a hematopoietic neoplasm. The neoplasm may be neurofibromatosis. In addition, the neoplasm may be metastatic.

The reovirus may be a mammalian or avian reovirus. In another embodiment, more than one type or strain of reovirus is used. A human reovirus may be used, for example, serotype 1 reovirus (Lang), serotype 2 reovirus (Jones), or serotype 3 reovirus (Dearing or Abney). The reovirus may also be one or more field isolates from one or more species, including but not limited to avian and mammalian species.

In another embodiment of the invention, the reovirus is one or more recombinant reoviruses. The recombinant reovirus may be from two or more strains of reovirus. The recombinant reovirus may be naturally occurring or non-naturally-occurring. The recombinant reovirus may comprise naturally occurring variant coat protein coding sequences or mutated coat protein coding sequences. In one embodiment, the recombinant reovirus results from reassortment of reoviruses selected from the group consisting of serotype 1 reovirus, serotype 2 reovirus, and serotype 3 reovirus. The recombinant reovirus may be generated by co-infection of mammalian cells with different subtypes of reoviruses.

In one embodiment of the invention, approximately 1 to approximately 10¹⁵ plaque-forming units (pfu) of reovirus/kg body weight is administered. The reovirus may be administered in a single dose or in more than one dose. The invention also contemplates a number of routes of administration for the invention. In one embodiment, the reovirus is administered by injection into or near the solid neoplasm. In another embodiment, the reovirus is administered, for example, intravascularly, intrathecally, intravenously, intramuscularly, subcutaneously, intraperitoneally, topically, orally, rectally, vaginally, nasally, or intratumorally. More than one route of administration may be used to deliver reovirus. In another embodiment, reovirus is administered along with an effective amount of a chemotherapeutic agent. The chemotherapeutic agent is preferably not 1,3-bis(2-chloroethyl)-1-nitrosourea (BCNU). The invention further includes the use of an appropriate immunosuppressive composition in combination with any reoviruses described herein.

The reovirus may be immunoprotected or encapsulated in a micelle. In addition, any reovirus or combination of reoviruses described herein may be otherwise chemically or genetically modified; for example, the reovirus may be treated with a protease prior to administration.

The present invention may optionally comprise the step of suppressing or otherwise inhibiting the immune system of the animal, which may be performed concurrently or subsequently with administration of the virus. The immune systems may be compromised by one or more of the following: an HIV infection; as a side effect of chemotherapy or radiation therapy; by selective removal of B and/or T cell populations; by removal of antibodies (anti-antireovirus antibodies or all antibodies), and the like. In another embodiment, the reovirus is administered along with an effective amount of an anti-antireovirus antibody.

The immunosuppression or immunoinhibition may be accomplished by means of an immunosuppressant, an immune suppressive agent, or by any other means which inhibits a mammal's immune system or renders the mammal immunodeficient. When an immunosuppressant is used, it is preferably administered prior to or concurrent with reovirus administration. The mammal may be rendered immunosuppressed, immunodeficient, or immunoinhibited prior to or concurrent with reovirus administration.

The invention may also be practiced with other oncolytic viruses in the same manner as with reovirus. In particular, oncolytic viruses that do not inhibit PKR function are preferred. More preferably, the virus is an adenovirus mutated in the VA1 region, a herpes virus mutated in the γ134.5 gene, a vaccinia virus mutated in the K3L and/or E3L region, a parapoxyims orf virus mutated in the OV20.0L gene, or an influenza virus mutated in the NS-1 gene.

The details of one or more embodiments of the invention are set forth in the accompanying drawings and the description below. Other features, objects, and advantages of the invention will be apparent from the description and drawings, and from the claims.

### DETAILED DESCRIPTION

The present invention provides a method of treating a neoplasm in an animal by using an oncolytic virus, particularly a reovirus, wherein the neoplasm has an activated Rac pathway or elevated PP2A-like phosphatase activities. Also provided are methods of determining if a neoplasm is susceptible to reovirus (or other oncolytic viruses) infection by measuring the activities of PP2A-like phosphatase or the Rac pathway.

Prior to describing the invention in further detail, the terms used in this application are defined as follows unless otherwise indicated.

Definitions

A “neoplastic cell”, “tumor cell”, or “cell with a proliferative disorder” refers to a cell which proliferates at an abnormally high rate. A new growth comprising neoplastic cells is a “neoplasm”, also known as a “tumor”. A tumor is an abnormal tissue growth, generally forming a distinct mass that grows by cellular proliferation more rapidly than normal tissue growth. A tumor may show partial or total lack of structural organization and functional coordination with normal tissue. As used herein, a tumor is intended to encompass hematopoietic tumors as well as solid tumors.

A tumor may be benign (benign tumor) or malignant (malignant tumor or cancer). Malignant tumors can be broadly classified into three major types. Malignant tumors arising from epithelial structures are called carcinomas; malignant tumors that originate from connective tissues such as muscle, cartilage, fat, or bone are called sarcomas; and malignant tumors affecting hematopoietic structures (structures pertaining to the formation of blood cells) including components of the immune system, are called leukemias and lymphomas. Other tumors include, but are not limited to neurofibromatosis.

A neoplasm with an “activated PP2A-like phosphatase” or “elevated PP2A-like phosphatase activities” is a neoplasm in which the neoplastic cells have abnormally high protein phosphatase 2A(PP2A)-like activities. Methods for assaying PP2A-like phosphatase activities are known in the art and are described in more detail below. Neoplastic cells have abnormally high PP2A-like phosphatase activities if, compared to non-neoplastic cells, the neoplastic cells have higher PP2A-like phosphatase activities. In particular, if the neoplastic cells have higher PP2A-like phosphatase activities in the absence of mitogens, such as serum, then the neoplastic cells have abnormally high PP2A-like phosphatase activities. The PP2A-like phosphatase activity of the neoplasm is preferably at least about 10%, more preferably about 20%, 30%, 40%, 50%, 60%, 70%, 80%, or 90% higher than that of non-neoplastic cells. Most preferably, the PP2A-like phosphatase activity of the neoplasm is at least about 100% higher than that of non-neoplastic cells (i.e., at least about twice as high as the PP2A-like phosphatase activity of non-neoplastic cells).

Abnormally high PP2A-like phosphatase activities can be caused by many reasons. For example, the PP2A phosphatase in the neoplastic cells may have a mutation that renders it constitutively activated, in particular a mutation in the regulatory subunit. It is also possible that an upstream element of the PP2A-like phosphatase is mutated, leading to abnormal activation of the PP2A-like phosphatase. In addition, factors that control the production or stability of PP2A or any of its upstream elements may also be mutated in such a manner as to increase the level of PP2A-like activities.

A neoplasm with an “activated Rac pathway” is a neoplasm in which the Rac pathway is more active than that in a non-neoplastic cell. In particular, the Rac pathway is constitutively active. The Rac pathway may be activated by way of rac gene mutation, elevated level of rac gene expression, elevated stability of the rac gene message, or any mutation or other mechanism which leads to the activation of Rac or an element or elements downstream or upstream from Rac in the Rac pathway, thereby increasing the Rac pathway activity. The Rac pathway is known in the art. For example, elements upstream from Rac include, without being limited to, Cdc42, Tiam1, Ras, Sos, Grb2, pI3 kinase, Lck, Vav, Eps8, and E3b1 (see, e.g., Scita et al., 2000). Elements downstream from Rac include, without being limited to, Rho and the p21-activated kinase (PAK). In addition, factors that control the production of Rac or any of its upstream or downstream elements may also be mutated in such a manner as to increase the level of Rac-pathway activities.

A “mutation” may be a deletion, insertion, or substitution of any nucleotide(s) or amino acid(s).

“Infection by reovirus” refers to the entry and replication of reovirus in a cell. Similarly, “infection of a tumor by reovirus” refers to the entry and replication of reovirus in the cells of the tumor.

“Reovirus” refers to any virus classified in the reovirus genus, whether naturally occurring, modified or recombinant. Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kbp. The individual RNA segments vary in size. Three distinct but related types of reovirus have been recovered from many species. All three types share a common complement-fixing antigen.

The human reovirus consists of three serotypes: type 1 (strain Lang or TiL), type 2 (strain Jones, T2J) and type 3 (strain Dearing or strain Abney, T3D). The three serotypes are easily identifiable on the basis of neutralization and hemagglutinin-inhibition assays (see, for example, Fields, B. N. et al., 1996).

The reovirus may be naturally occurring or modified. The reovirus is “naturally occurring” when it can be isolated from a source in nature and has not been intentionally modified by humans in the laboratory. For example, the reovirus can be from a “field source”, that is, from a human who has been infected with the reovirus.

The reovirus may be modified but still capable of lytically infecting a mammalian cell having an active ras pathway. The reovirus may be chemically or biochemically pretreated (e.g., by treatment with a protease, such as chymotrypsin or trypsin) prior to administration to the proliferating cells. Pretreatment with a protease removes the outer coat or capsid of the virus and may increase the infectivity of the virus. The reovirus may be coated in a liposome or micelle (Chandran and Nibert, 1998). For example, the virion may be treated with chymotrypsin in the presence of micelle forming concentrations of alkyl sulfate detergents to generate a new infectious subvirion particle.

The reovirus may be a recombinant (i.e., reassorted) reovirus resulting from the recombination/reassortment of genomic segments from two or more genetically distinct reoviruses. Recombination/reassortment of reovirus genomic segments may occur in nature following infection of a host organism with at least two genetically distinct reoviruses. Recombinant virions can also be generated in cell culture, for example, by co-infection of permissive host cells with genetically distinct reoviruses (Nibert et al. 1995).

Accordingly, the invention contemplates the use of a recombinant reovirus resulting from reassortment of genome segments from two or more genetically distinct reoviruses, including but not limited to, human reovirus, such as type 1 (e.g., strain Lang), type 2 (e.g., strain Jones), and type 3 (e.g., strain Dearing or strain Abney), non-human mammalian reoviruses, or avian reovirus. The invention further contemplates the use of recombinant reoviruses resulting from reassortment of genome. segments from two or more genetically distinct reoviruses wherein at least one parental virus is genetically engineered, comprises one or more chemically synthesized genomic segment, has been treated with chemical or physical mutagens, or is itself the result of a recombination event. The invention further contemplates the use of the recombinant reovirus that has undergone recombination in the presence of chemical mutagens, including but not limited to dimethyl sulfate and ethidiurh bromide, or physical mutagens, including but not limited to ultraviolet light and other forms of radiation.

The invention further contemplates the use of recombinant reoviruses that comprise deletions or duplications in one or more genome segments, that comprise additional genetic information as a result of recombination with a host cell genome, or that comprise synthetic genes.

The reovirus may be modified by incorporation of mutated coat proteins, such as for example, into the virion outer capsid. The proteins may be mutated by replacement, insertion, or deletion. Replacement includes the insertion of different amino acids in place of the native amino acids. Insertions include the insertion of additional amino acid residues into the protein at one or more locations. Deletions include deletions of one or more amino acid residues in the protein. Such mutations may be generated by methods known in the art. For example, oligonucleotide site directed mutagenesis of the gene encoding for one of the coat proteins could result in the generation of the desired mutant coat protein. Expression of the mutated protein in reovirus infected mammalian cells in vitro such as COS 1 cells will result in the incorporation of the mutated protein into the reovirus virion particle (Turner and Duncan, 1992; Duncan et al., 1991; Mah et al., 1990).

The reovirus is preferably a reovirus modified to reduce or eliminate an immune reaction to the reovirus. Such a modified reovirus is termed “immunoprotected reovirus”. Such modifications could include packaging of the reovirus in a liposome, a micelle, or other vehicle to mask the reovirus from the immune system. Alternatively, the outer capsid of the reovirus virion particle may be removed since the proteins present in the outer capsid are the major determinant of the host humoral and cellular responses.

An “immunoprotected virus” is a virus modified to reduce or eliminate an immune reaction to the virus. The modifications could include packaging of the virus in a liposome, a micelle, or other vehicle to mask the virus from the host immune system. Alternatively, the outer capsid of the virus virion particle may be removed since the proteins present in the outer capsid are the major determinant of the host humoral and cellular responses. In addition to reducing or eliminating immune responses, the modifications may also reduce non-specific uptake of the virus in normal tissues.

An “oncolytic virus” is a virus that preferentially replicates in, and kills, neoplastic cells. An oncolytic virus may be a naturally occurring virus or an engineered virus. Oncolytic viruses also encompass immunoprotected and reassortant viruses as described in detail for reovirus. The virus is “naturally occurring” when it can be isolated from a source in nature and has not been intentionally modified by humans in the laboratory. For example, the virus can be from a “field source”, that is, from an infected animal. The virus is “engineered” when it has been modified by human intervention. For example, the genetic material of the virus may be mutated, or the virus particle may be modified.

“Administration” of a virus to a subject refers to the act of administering the virus to a subject in a manner so that it contacts the target neoplastic cells. The route by which the virus is administered, as well as the formulation, carrier or vehicle, will depend on the location as well as the type of the target cells.

The term “substantial lysis” means at least about 10% of the cells of a neoplasm are lysed. More preferably, at least about 20%, 30%, 40%, 50%, 60%, 70%, 80% or 90% of the cells are lysed. Most preferably, at least about 95% of the cells are lysed. The percentage of lysis can be determined, for example, by measuring the reduction in the size of the tumor or reduction of symptoms of the tumor.

A “mammal suspected of having a neoplasm” is a mammal that has a genetic disposition for a tumor, or a mammal in which the tumor or substantially all of the tumor has been surgically removed but is suspected of harboring residual tumor cells.

“Treating or alleviating a tumor” means alleviating or eliminating the symptoms of a tumor, or slowing down the progress of the tumor. The alleviation is preferably at least about 10%, more preferably at least about 20%, 30%, 40%, 50%, 60%, 70%, 80%, 90% or 95%.

A “metastatic tumor” is a tumor that has metastasized from a tumor located at another place in the same animal.

An “effective amount” is an amount of an agent that is sufficient to result in the intended effect. For an oncolytic virus used to treat or ameliorate a tumor, an effective amount is an amount of the virus sufficient to alleviate or eliminate the symptoms of the tumor or to slow down the progress of the tumor.

The terms “immunosuppressant” or “immune suppressive agent” include conventional immunosuppressants, immunoinhibitors, antibodies, and conditions such as radiation therapy or HIV infection which result in compromise of the immune system.

Methods

Reovirus is an effective therapeutic agent against ras-activated neoplasia because it selectively replicates in cells with an activated ras pathway (U.S. Pat. No. 6,136,307). The ras pathway is not activated in normal cells; therefore, reovirus kills neoplastic cells with high selectivity. Without being limited to a theory, it is thought that viral gene transcription in normal cells correlated with phosphorylation of a cellular protein of approximately 65 kDa, determined to be double-stranded RNA-activated protein kinase (PKR), that was not observed in ras-activated cells. Phosphorylation of PKR leads to inhibition of translation; therefore, viral replication cannot be completed. In ras-activated cells, however, ras or its downstream factors blocks the phosphorylation of PKR, thereby allowing viral translation and replication to go on.

We have now found that the lack of PKR phosphorylation in transformed cells is due to the activation of a PP2A-like phosphatase in these cells. Accordingly, reovirus can be used to infect and kill neoplastic cells that have abnormally high activities of PP2A. In addition, the PP2A-like phosphatase activity can also serve as a basis of diagnosis for neoplastic cells, particularly neoplastic cells that are susceptible to reovirus infection.

PP2A, or protein phosphatase type 2A, is a serine-threonine phosphoprotein phosphatase. There are four major types of serine-threonine phosphoprotein phosphatases: 1, 2A, 2B and 2C. All of these phosphatases consist of a catalytic subunit, which is homologous among different types, and at least one regulatory subunit. By dephosphorylating proteins that have been phosphorylated by protein kinase A, these phophatases keep the functions of protein kinase A to a transient, and regulatory, role.

PP2A-like phosphatase activities can be determined by any method known in. the art. For example, PP2A-like activities can be determined as the difference between total phosphatase activities and phosphatase activities in the presence of low concentration of okadaic acid (Toumebize et al., 1997; Mills et al., 1998; Tian et al., 1998). It has been reported that okadaic acid is the most effective against PP2A, less so against PP1, and only marginally against PP2B (Bialojan et al., 1988). Since PP2C is relatively rare, okadaic-sensitive serine-threonine phosphatase activity can thus be deemed PP2A-like activity. The concentration of okadaic acid to be employed in this assay may vary with the source and content of the sample. For example, high protein content may alter the inhibitory effect of okadaic acid. However, the concentration of okadaic acid is preferably between about 0.1 nM and 500 nM, more preferably between about 0.2 nM and 100 nM, and most preferably between about 0.2 nM and 10 nM. To ascertain that an appropriate concentration is used, purified PP2A and PP1 can be spiked into the samples to serve as controls. A concentration of okadaic acid that selectively inhibits PP2A but not PP 1 can thus be determined.

Elements immediately downstream of Ras have also been investigated as to their roles in reovirus infection. Using a variety of effector domain mutant of Ras, we have found that Ral and Rac are exploited by reovirus for its own replication. Thus, as cells transformed with activated Ras mutants were susceptible to reovirus infection, cells harboring Ras mutants that specifically interact with Ral or Rac were also susceptible. These results indicate that cells with an activated Ral or Rac pathway can be lysed by reovirus. Accordingly, reovirus can be used to treat neoplasms comprising an activated Rac pathway. Alternatively, the activity of the Rac pathway, or an analysis of whether any of the Rac pathway elements is mutated, may be used to diagnose neoplasms, in particular susceptibility of neoplasms to reovirus. The activity of the Rac pathway can be determined, for example, by measuring the activity of Rac, Rho, or PAK according to any method established in the art.

Rac can be activated by factors outside of the ras pathway. It is contemplated that the present invention can be used to treat neoplasms that do not have any mutation in the ras gene or its upstream elements (such as EGFR or PDGFR).

Various reoviruses can be used to practice the invention. Representative types of human reovirus include type 1 (e.g., strain Lang or T1L); type 2 (e.g., strain Jones or T2J); and type 3 (e.g., strain Dearing or strain Abney, T3D or T3A). In a preferred embodiment, the reovirus is human reovirus serotype 3. More preferably the reovirus is human reovirus serotype 3, strain Dearing. Alternatively, the reovirus can be a non-human mammalian reovirus (e.g., a non-human primate reovirus, such as baboon; equine; or canine reovirus) or a non-mammalian reovirus (e.g., avian reovirus). A combination of different serotypes and/or different strains of reovirus, such as reovirus from different species of animal, can be used.

The reovirus may be naturally occurring or modified. The reovirus may be modified but still capable of lytically infecting an animal cell having an activated ras pathway. The reovirus may be chemically or biochemically pretreated (e.g., by treatment with a protease, such as chyrnotrypsin or trypsin) prior to administration to the proliferating cells. Pretreatment with a protease removes the outer coat or capsid of the virus and may increase the infectivity of the virus. The reovirus may be coated in a liposome or micelle (Chandran and Nibert, 1998) to reduce or prevent an immune response from a mammal which has developed immunity to the reovirus. For example, the virion may be treated with chymotrypsin in the presence of micelle-forming concentrations of alkyl sulfate detergents to generate a new infectious subvirion particle.

The reovirus may be a recombinant reovirus resulting from the recombination/reassortment of genomic segments from two or more genetically distinct reoviruses. Recombination/reassortment of reovirus genomic segments may occur in nature following infection of a host organism with at least two genetically distinct reoviruses. Recombinant virions can also be generated in cell culture, for example, by co-infection of permissive host cells with genetically distinct reoviruses (Nibert et al. 1995).

Accordingly, the invention contemplates the use of recombinant reoviruses resulting from reassortment of genome segments from two or more genetically distinct reoviruses, including but not limited to, human reovirus, such as type 1 (e.g., strain Lang), type 2 (e.g., strain Jones), and type 3 (e.g., strain Dearing or strain Abney), non-human mammalian reoviruses, or avian reovirus. The invention further contemplates the use of recombinant reoviruses resulting from reassortment of genome segments from two or more genetically distinct reoviruses wherein at least one parental virus is genetically engineered, comprises one or more chemically synthesized genomic segment, has been treated with chemical or physical mutagens, or is itself the result of a recombination event. The invention further contemplates the use of recombinant reovirus that have undergone recombination in the presence of chemical mutagens, including but not limited to dimethyl sulfate and ethidium bromide, or physical mutagens, including but not limited to ultraviolet light and other forms of radiation.

The invention further contemplates the use of recombinant viruses that comprise deletions or duplications in one or more genome segments, that comprise additional genetic information as a result of recombination with a host cell genome, or that comprise synthetic genes.

The reovirus may be modified by incorporation of mutated coat proteins, such as for example, into the virion outer capsid. The proteins may be mutated by replacement, insertion, or deletion. Replacement includes the insertion of different amino acids in place of the native amino acids. Insertions include the insertion of additional amino acid residues into the protein at one or more locations. Deletions include deletions of one or more amino acid residues in the protein. Such mutations may be generated by methods known in the art. For example, oligonucleotide site directed mutagenesis of the gene encoding for one of the coat proteins could result in the generation of the desired mutant coat protein. Expression of the mutated protein in reovirus infected mammalian cells in vitro such as COS1 cells will result in the incorporation of the mutated protein into the reovirus virion particle (Turner et al., 1992; Duncan et al., 1991; Mah et al., 1990).

The reovirus is preferably an immunoprotected reovirus. The modifications could include packaging of the reovirus in a liposome, a micelle, or other vehicle to mask the reovirus from the host immune system. Alternatively, the outer capsid of the reovirus virion particle may be removed since the proteins present in the outer capsid are the major determinant of the host humoral and cellular responses. In addition to reducing or eliminating immune responses, the modifications may also reduce non-specific uptake of the virus in normal tissues. As discussed above, reovirus is capable of binding to a multitude of cell types, presumably due to the ubiquitous nature of its receptor. Therefore, by masking the reovirus, non-specific binding and uptake can be reduced.

In addition to reovirus, other oncolytic viruses can be used to practice the present invention in the same manner as reoviruses. In particular, oncolytic viruses that do not inhibit PKR function are preferred. These viruses may be naturally existing, like the reovirus, or they may be modified or mutated such that a viral factor that inhibits PKR is not functional.

A few such oncolytic viruses are discussed below, and a person of ordinary skill in the art can practice the present invention using additional oncolytic viruses as well according to the disclosure herein and knowledge available in the art. The oncolytic virus may be a member in the family of myoviridae, siphoviridae, podoviridae, teciviridae, corticoviridae, plasmaviridae, lipothrixviridae, fuselloviridae, poxyiridae, iridoviridae, phycodnaviridae, baculoviridae, herpesviridae, adenoviridae, papovaviridae, polydnaviridae, inoviridae, microviridae, geminiviridae, circoviridae, parvoviridae, hepadnaviridae, retroviridae, cyctoviridae, reoviridae, bimaviridae, paramyxoviridae, rhabdoviridae, filoviridae, orthomyxoviridae, bunyaviridae, arenaviridae, leviviridae, picomaviridae, sequiviridae, comoviridae, potyviridae, caliciviridae, astroviridae, nodaviridae, tetraviridae, tombusviridae, coronaviridae, glaviviridae, togaviridae, or barnaviridae. As with reovirus, immunoprotected or reassortant viruses of other oncolytic viruses are also encompassed in the present invention. Furthermore, a combination of at least two oncolytic viruses, including reovirus, can also be employed to practice the present invention.

Normally, when virus enters a cell, double stranded RNA Kinase (PKR) is activated and blocks protein synthesis, and the virus cannot replicate in this cell. Some viruses have developed a system to inhibit PKR and facilitate viral protein synthesis as well as viral replication. For example, adenovirus makes a large amount of a small RNA, VA1 RNA. VA1 RNA has extensive secondary structures and binds to PKR in competition with the double stranded RNA (dsRNA) which normally activates PKR. Since it requires a minimum length of dsRNA to activate PKR, VA1 RNA does not activate PKR. Instead, it sequesters PKR by virtue of its large amount. Consequently, protein synthesis is not blocked, and adenovirus can replicate in the cell.

Neoplastic cells that have an activated PP2A-like phosphatase or Rac pathway are not subject to protein synthesis inhibition by PKR, because PKR is inhibited in these cells. These neoplastic cells are therefore susceptible to viral infection even if the virus does not have a PKR inhibitory system. Accordingly, if the PKR inhibitors in adenovirus is mutated so as not to block PKR function anymore, the resulting virus does not infect normal cells due to protein synthesis inhibition by PKR, but it can replicate in neoplastic cells which lack PKR activities.

Accordingly, a virus that is modified or mutated such that it does not inhibit PKR function selectively replicates in neoplastic cells with an activated PP2A-like phosphatase or Rac pathway while normal cells are resistant. Preferably, the virus is an adenovirus mutated in the VA1 region, a herpes virus mutated in the γ134.5 gene, a vaccinia virus mutated in the K3L and/or E3L region, a parapoxvirus orfvirus mutated in the OV20.0L gene, or an influenza virus mutated in the NS-1 gene.

The viruses can be modified or mutated according to the known structure-function relationship of the viral PKR inhibitors. For example, since the amino terminal region of E3 protein interacts with the carboxy-terminal region domain of PKR, deletion or point mutation of this domain prevents anti-PKR function (Chang et al., 1992, 1993, 1995; Sharp et al., 1998; Romano et al., 1998). The K3L gene of vaccinia virus encodes pK3, a pseudosubstrate of PKR. There is a loss-of-function mutation within K3L. Truncations or point mutations within the C-terminal portion of K3L protein that is homologous to residues 79 to 83 in eIF-2 abolish PKR inhibitory activity (Kawagishi-Kobayashi et al., 1997).

Neoplasms with an activated PP2A-like phosphatase or Rac pathway may have any histological or anatomical properties. The neoplasms may include, for example, forms of breast cancer, central nervous system cancer (e.g., neuroblastoma and glioblastoma), peripheral nervous system cancer, lung cancer, prostate cancer, colorectal cancer, thyroid cancer, renal cancer, adrenal cancer, liver cancer, lymphoma, and leukemia. Of particular interest are forms of cancer in which ras mutations are rare, for example, lymphoid malignancies, including diffuse large B-cell lymphomas (Nedergaard et al., 1997 and references within).

The route by which the virus is administered, as well as the formulation, carrier or vehicle, will depend on the location as well as the type of the neoplasm. A wide variety of administration routes can be employed. For example, for a solid neoplasm that is accessible, the virus can be administered by injection directly to the neoplasm. For a hematopoietic neoplasm, for example, the virus can be administered intravenously or intravascularly. For neoplasms that are not easily accessible within the body, such as metastases or brain tumors, the virus is administered in a manner such that it can be transported systemically through the body of the mammal and thereby reach the neoplasm (e.g., intrathecally, intravenously, or intramuscularly). Alternatively, the virus can be administered directly to a single solid neoplasm, where it then is carried systemically through the body to metastases. The virus can also be administered subcutaneously, intraperitoneally, topically (e.g., for melanoma), orally (e.g., for oral or esophageal neoplasm), rectally (e.g., for colorectal neoplasm), vaginally (e.g., for cervical or vaginal neoplasm), nasally or by inhalation (e.g., for lung neoplasm).

The virus can be administered systemically to mammals which are immune compromised or which have not developed immunity to the virus. In such cases, viruses that are administered systemically, i.e., by intravenous injection, will spread to the locations of the neoplastic cells, resulting in lysis of the cells.

Immune Suppression

Immunocompetent mammals previously exposed to a virus subtype may have developed humoral and/or cellular immunity to that virus subtype. Nevertheless, it has been found that direct injection of reovirus into a solid tumor in immunocompetent mammals will result in the lysis of the neoplastic cells.

On the other hand, when the virus is administered systemically to immuriocompetent mammals, the mammals may produce an immune response to the virus. Although systemic administration of reovirus has been shown to successfully lead to oncolysis of local tumors in immunocompetent animals, it is preferable to avoid immune responses against the virus, particularly in animals that have previously received large amounts of the same virus. Immune responses may be avoided if the virus is of a subtype to which the mammal has not developed immunity, or if the virus has been modified as previously described herein such that it is immunoprotected, for example, by protease digestion of the outer capsid or packaging in a micelle.

Alternatively, it is contemplated that the immunocompetency of the mammal against the virus may be suppressed either by the co-administration of pharmaceuticals known in the art to suppress the immune system in general (Cuff et al., 1998) or alternatively by administration of anti-idiotypic antibodies that recognize the antibodies for that virus. The humoral immunity of the mammal against virus may also be temporarily reduced or suppressed by plasmaphoresis of the mammals blood to remove antibodies specific for that virus. The humoral immunity of the mammal against the virus may additionally be temporarily reduced or suppressed by the intraveneous administration of non-specific immunoglobulin to the mammal. The immune system may also be suppressed by anti-CD4 and/or anti-CD8 antibodies, or complement neutralization.

It is contemplated that the virus may be administered to immunocompetent mammals in conjunction with the administration of immunosuppressants and/or immunoinhibitors. Such immunosuppressants and immunoinhibitors are known to those of skill in the art and include such agents as cyclosporin, rapamycin, tacrolimus, mycophenolic acid, azathioprine and their analogs, and the like. Other agents are known to have immunosuppressant properties as well (see, e.g., Goodman and Gilman, 7th Edition, page 1242, the disclosure of which is incorporated herein by reference).

Such immunoinhibitors also include anti-antivirus antibodies, which are antibodies directed against anti-virus antibodies that specifically recognize the virus of interest. Such antibodies can be made by methods known in the art. See for example “Antibodies: A laboratory manual” E. Harlow and D. Lane, Cold Spring Harbor Laboratory, 1988). Such anti-antivirus antibodies may be administered prior to, at the same time, or shortly after the administration of the virus. Preferably an effective amount of the anti-antivirus antibodies are administered in sufficient time to reduce or eliminate an immune response by the mammal to the administered virus.

Alternatively or in addition, T cells and/or B cells, or subsets thereof, can be selectively removed from the animal, for example by administration of anti-CD4 and/or anti-CD8 antibodies.

Compositions

This invention also includes pharmaceutical compositions which contain, as the active ingredient, one or more of the reoviruses associated with pharmaceutically acceptable carriers or excipients. The invention further includes pharmaceutical compositions which contain, as the active ingredient, one or more of the reoviruses, along with an appropriate immunosuppresant, associated with pharmaceutically acceptable carriers or excipients. In making the compositions of this invention, the active ingredient/reovirus is usually mixed with an excipient, diluted by an excipient, or enclosed within such a carrier which can be in the form of a capsule, sachet, paper or other container. When the pharmaceutically acceptable excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid compositions such as tablets, the principal active ingredient/reovirus is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills, and capsules.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device, or the nebulizing device may be attached to a face mask tent or intermittent positive pressure breathing machine. Solution, suspension, or powder compositions may be administered, preferably orally or nasally, from devices which deliver the formulation in an appropriate manner.

Another preferred formulation employed in the methods of the present invention employs transdermal delivery devices (“patches”). Such transdermal patches may be used to provide continuous or discontinuous infusion of the reovirus of the present invention in controlled amounts. The construction and use of transdermal patches for the delivery of pharmaceutical agents is well known in the art. See, e.g., U.S. Pat. No. 5,023,252, herein incorporated by reference. Such patches may be constructed for continuous, pulsatile, or on-demand delivery of pharmaceutical agents.

Other suitable formulations for use in the present invention can be found in Remington's Pharmaceutical Sciences.

The reovirus or the pharmaceutical composition comprising the reovirus may be packaged into convenient kits providing the necessary materials packaged into suitable containers. It is contemplated that the kits may also include chemotherapeutic agents and/or anti-antireovirus antibody.

The reovirus is administered in an amount that is sufficient to treat the neoplasm (e.g., an “effective amount”). A neoplasm is “treated” when administration of reovirus to the proliferating cells effects lysis of the proliferating cells. This may result in a reduction in size of the neoplasm or a complete elimination of the neoplasm. The reduction in size of the neoplasm, or elimination of the neoplasm, is generally caused by lysis of neoplastic cells (“oncolysis”) by the reovirus. Preferably the effective amount is that amount able to inhibit tumor cell growth. Preferably the effective amount is from about 1.0 pfu/kg body weight to about 10¹⁵ pfu/kg body weight, and more preferably from about 102 pfu/kg body weight to about 10¹³ pfu/kg body weight. For example, for treatment of a human, approximately 10² to 10¹⁷ pfU of reovirus can be used, depending on the type, size, and number of tumors present. The effective amount will be determined on an individual basis and may be based, at least in part, on consideration of the type of reovirus; the chosen route of administration; the individual's size, age, gender; the severity of the patient's symptoms; the size and other characteristics of the neoplasm; and the like. The course of therapy may last from several days to several months or until diminution of the disease is achieved.

The reovirus can be administered in a single dose, or multiple doses (i.e., more than one dose). The multiple doses can be administered concurrently, or consecutively (e.g., over a period of days or weeks). The reovirus can also be administered to more than one neoplasm in the same individual.

The compositions are preferably formulated in a unit dosage form, each dosage containing from about 10² pfus to about 10¹³ pfu of the reovirus. The term “unit dosage forms” refers to physically discrete units suitable as unitary dosages for human subjects and other mammals, each unit containing a predetermined quantity of reovirus calculated to produce the desired therapeutic effect, in association with a suitable pharmaceutical excipient.

It has been found that the reovirus is effective for the treatment of solid neoplasms in immunocompetent mammals. Administration of unmodified reovirus directly to the neoplasm results in oncolysis of the neoplastic cells and reduction in the size of the tumor.

It is contemplated that the reovirus may be administered in conjunction with surgery or removal of the neoplasm. Therefore, provided herewith are methods for the treatment of a solid neoplasm comprising surgical removal of the neoplasm and administration of a reovirus at or near to the site of the neoplasm.

It is contemplated that the reovirus may be administered in conjunction with or in addition to radiation therapy.

It is further contemplated that the reovirus of the present invention may be administered in conjunction with or in addition to one or more known anticancer compounds or chemotherapeutic agents. Chemotherapeutic agents are compounds which may inhibit the growth of tumors. Such agents, include, but are not limited to, 5-fluorouracil, mitomycin C, methotrexate, hydroxyurea, cyclophosphamide, dacarbazine, mitoxantrone, anthracyclins (Epirubicin and Doxurubicin), antibodies to receptors, such as herceptin, etopside, pregnasome, platinum compounds such as carboplatin and cisplatin, taxanes such as taxol and taxotere, hormone therapies such as tamoxifen and anti-estrogens, interferons, aromatase inhibitors, progestational agents and LHRH analogs.

Preferably the reovirus is administered in the absence of 1,3-bis(2-chloroethyl)-1-nitrosourea (BCNU). For example, the 1,3-bis(2-chloroethyl)-1-nitrosourea (BCNU) is not administered to the mammal either before, during, or after the mammal receives the reovirus.

The reoviruses of the present invention have been found to reduce the growth of tumors that are metastatic. In an embodiment of the invention, a method is provided for reducing the growth of metastastic tumors in a mammal comprising administering an effective amount of a reovirus to the mammal.

In order to further illustrate the present invention and advantages thereof, the following specific examples are given but are not meant to limit the scope of the claims in any way.

### EXAMPLES

In the examples below, all temperatures are in degrees Celsius (unless otherwise indicated) and all percentages are weight percentages (also unless otherwise indicated).

In this application, the following abbreviations have the following meanings. If an abbreviation is not defined, it has its generally accepted meaning:

| | |
|---|---|
| μM = | micromolar |
| mM = | millimolar |
| M = | molar |
| ml = | milliliter |
| μl = | microliter |
| mg = | milligram |
| μg = | microgram |
| FBS = | fetal bovine serum |
| DTT = | dithiothrietol |
| SDS = | sodium dodecyl sulfate |
| PBS = | phosphate buffered saline |
| DMEM = | Dulbecco's modified Eagle's medium |
| MEM = | modified Eagle's medium |
| b-ME = | b-mercaptoethanol |
| MOI = | multiplicity of infection |
| PFU or pfu = | plaque forming units |
| MAPK = | MAP kinase |
| HRP = | horseradish-peroxidase |
| PKR = | double-stranded RNA activated protein kinase |
| RT-PCR = | reverse transcriptase-polymerase chain reaction |
| GAPDH = | glyceraldehyde-3-phosphate dehydrogenase |
| EGFR = | epidermal growth factor receptors |
| SCID = | severe combined immunodeficiency |
| PP2A = | protein phosphatase type 2A |
| BCNU = | 1,3-bis(2-chloroethyl)-1-nitrosourea |

### Example 1

### PP2A-Like Phosphatase Inactivates PKR

PKR is not phosphorylated (i.e., active) in cells transformed with elements of the ras pathway, such as v-erbB. To determine whether serine-threonine phosphatases are responsible for the lack of phosphorylation of PKR in these cells, selective inhibitors of the phosphatases are employed to assess the role of each phosphatase. The result indicates that in the presence of okadaic acid, a selective inhibitor of PP2A-like phosphatase, PKR becomes phosphorylated. Furthermore, the okadaic acid-treated cells also become susceptible to reovirus infection. Inhibitors specific for PP1 or PP2C have no effects on PKR phosphorylation.

The PP2A-like phosphatase activities in untransformed NIH3T3 cells and NIH3T3 cells transformed with v-erbB are also determined. Since PP2A is known to regulate the cell cycle, PP2A-like activities vary with the status of a cell in the cell cycle. Therefore, cells from several different flasks are pooled to ensure that they are not synchronized. The ability of the cell lysate from transformed or untransformed cells to de-phosphorylate a substrate (see, e.g., Tournebize et al., 1997; Mills et al., 1998; Tian et al., 1998) is then measured in the presence or absence of 10 nM okadaic acid. The results indicate that the transformed cells have significantly higher PP2A-like phosphatase activities than the untransformed cells.

Thus, the level of PP2A-like phosphatase activity negatively correlates with phosphorylation, or activation, of PKR. Activation of PKR, in turn, inhibits reovirus infection. Accordingly, neoplasms with abnormally high PP2A-like activities can be treated or alleviated by using reovirus or similar oncolytic viruses.

### Example 2

### Use of Cells Harboring ras Variants to Identify Downstream Effecters Associated with Reovirus Susceptibility

To determine the downstream signaling events important for reovirus-mediated oncolysis, a panel of variant Ras-transformed-NIH-3T3 cells was assayed for susceptibility to reovirus infection. The panel consisted of cells transformed with Ras variants harboring functionally distinct mutations in the effector-binding domain which causes selective interaction of the ras variants with only a subset of downstream ras effecters. Reovirus infections and assays for reovirus replication were performed essentially as described in U.S. Pat. No. 6,136,307, incorporated by reference in its entirety.

The results of these experiments show that cells transformed with the Ras variant that selectively interacts with Rac were susceptible to reovirus infection. In addition, cells harboring RasV12G37, which retains the ability to activate RalGEFs while lacking the ability to activate Raf or PI3-kinase, were also susceptible to reovirus infection. These data thus suggest that Rac and RalGEF are the downstream effecters responsible for the susceptibility of Ras-transformed cells to reovirus infection. Thus, activation of the Rac or ral pathway is sufficient to render cells susceptible to reovirus infection. Since the Rac or ml pathway can be activated by elements other than ras, these results also indicate that reovirus, or other similar oncolytic viruses, can be used to treat or alleviate neoplasms that have normal ras levels or activities, in addition to neoplasms having elevated ras levels or activities. Similarly, neoplasms that have normal levels or activities of the upstream elements of the ras pathway, such as EGFR or PDGFR, may also be treated or alleviated by these oncolytic viruses.

A number of embodiments of the invention have been described. Nevertheless, it will be understood that various modifications may be made without departing from the spirit and scope of the invention.

1. A method of treating or ameliorating a neoplasm in an animal, comprising administering to the animal an effective amount of one or more reoviruses under conditions which result in substantial lysis of neoplastic cells in the neoplasm, wherein the neoplasm comprises an activated PP2A-like phosphatase or an activated Rac pathway.

2. The method of claim 1, wherein the animal is a mammal.

3. The method of claim 2, wherein said mammal is selected from the group consisting of dogs, cats, sheep, goats, cattle, horses, pigs, humans, and non-human primates.

4. The method of claim 1 wherein the animal is a human.

5. The method of claim 1, wherein the reovirus is selected from the group consisting of mammalian reoviruses and avian reoviruses.

6. The method of claim 1, wherein the reovirus is a human reovirus.

7. The method of claim 6, wherein the reovirus is selected from the group consisting of serotype 1 reovirus, serotype 2 reovirus, and serotype 3 reovirus.

8. The method of claim 1, wherein more than one type of reovirus is administered.

9. The method of claim 1, wherein more than one strain of reovirus is administered.

10. The method of claim 1, wherein at least one of the reoviruses is a recombinant reovirus.

11. The method of claim 1, wherein about 1 to about 1015 plaque-forming units of reovirus/kg body weight are administered.

12. The method of claim 1, wherein the reovirus is administered in a single dose.

13. The method of claim 1, wherein the reovirus is administered in more than one dose.

14. The method of claim 1, wherein the neoplasm is a solid neoplasm.

15. The method of claim 14, wherein the neoplasm is selected from the group consisting of lung cancer, prostate cancer, colorectal cancer, thyroid cancer, renal cancer, adrenal cancer, liver cancer, pancreatic cancer, breast cancer, and central and peripheral nervous system cancer.

16. The method of claim 1, wherein the neoplasm is a hematopoietic neoplasm.

17. The method of claim 1, wherein the neoplasm is metastatic.

18. The method of claim 1, wherein the neoplasm is neurofibromatosis.

19. The method of claim 1, wherein the reovirus is administered by a route selected from the group consisting of intravascular, intrathecal, intravenous, intramuscular, subcutaneous, intraperitoneal, topical, oral, rectal, vaginal, nasal, and intratumoral administration.

20. The method of claim 1, wherein the animal is immunocompetent.

21. The method of claim 1, wherein the reovirus is immunoprotected.

22. The method of claim 1, wherein the reovirus is encapsulated in a micelle.

23. The method of claim 1, wherein the reovirus is treated with a protease prior to administration.

24. The method of claim 1, wherein the animal also receives an effective amount of an anti-antireovirus antibody.

25. The method of claim 1, further comprising the administration of an effective amount of a chemotherapeutic agent.

26. The method of claim 1, wherein the neoplastic cells comprise a normal ras gene.
